(19) United States
(12) Patent Application Publication
(10) Pub. No.: US 2025/0327122 A1
(43) Pub. Date: Oct. 23, 2025

(54) POLYMER SEQUENCING APPARATUS, METHODS OF FABRICATION AND USE

(71) Applicant: Armonica Technologies, Inc., Albuquerque, NM (US)

(72) Inventors: Victor Esch, Albuquerque, NM (US); Steve Brueck, Albuquerque, NM (US); Xin Jin, Albuquerque, NM (US); Hui Xia, Albuquerque, NM (US)

(21) Appl. No.: 19/255,318

(22) Filed: Jun. 30, 2025

Related U.S. Application Data
(63) Continuation of application No. 19/110,571, filed on Jan. 1, 0001.
(60) Provisional application No. 63/624,403, filed on Jan. 24, 2024.

(51) Int. Cl. C12Q 1/6869; G01N 21/65; G01N 27/447

(52) U.S. Cl. CPC C12Q 1/6869; G01N 21/65; G01N 27/44721; G01N 27/44791

(57) ABSTRACT

The systems disclosed herein may a nanoscale chip. The chip includes a nanochannel with two nanoscale transverse dimensions and a SERS enhancement structure therein, alignment marks for enabling positioning of a laser excitation beam onto the enhancement structure, and a structural element for controlling the positioning of a polymer within the nanofluidic chip relative to the enhancement structure. The system may further comprise a reader for analyzing polymers on the chip. The disclosure also relates to methods of fabricating the chip and sequencing a polymer using the chip and reader.

### TECHNICAL FIELD

The invention generally relates to materials and methods for sequencing polymers. In some embodiments, the materials and methods utilize Raman spectroscopy to identify polymer units. In some embodiments, the materials and methods utilize surface-enhanced Raman spectroscopy (SERS) enhancement structures within a nanofluidic chip.

### BACKGROUND

DNA is a long chain polymer composed of four nucleotides (Adenine, Cytosine, Guanine and Thymine) bound to a sugar-phosphate backbone. DNA can be either single stranded (ssDNA) or double-stranded (dsDNA). dsDNA typically contains two polymer chains (ssDNA) of corresponding nucleotides. The sequence of the bases in DNA contains the information (coding) for all know organisms and for many viruses. In some applications, sequencing one strand of ssDNA will provide an expected sequence of the corresponding ssDNA chain.

In addition to the four letters A, C, G and T, there are many epigenetic modifications that have profound effects on the outcomes of biological processes. The most well-known of these is 5-methylcytosine where an additional CH₃ (methyl) group is attached to the 5-position nitrogen of the cytosine ring. There are numerous of these “non-canonical” nucleotides that individually and collectively have important impacts on biology.

The human genome has approximately 6 billion nucleotides (or 3 billion base pairs in double stranded DNA, dsDNA). The sequences of the human genome include multiple repeating sections and epigenetic modifications. A 2017 review summarized both the enormous successes of current approaches and perspectives on the goals of further developments: “Nearly all of the aforementioned platforms require template amplification. However, the downsides of amplification include copying errors, sequence dependent biases and information loss (for example, methylation), not to mention added time and complexity. In an ideal world, sequencing would be native, accurate and without read length limitation.” See Shendure et al., “DNA sequencing at 40: past, present and future,” Nature 550, 345-353 (2017).

Massively parallel sequencing by synthesis (SBS), a predominant current technique from Illumina, relies on large numbers of short reads of 100 to 300 bases followed by an algorithmic reconstruction of the original genome sequence. Information on methylation is obtained by double reads with intermediate chemical modifications (e.g., bisulfate treatments) to selectively convert either modified or unmodified bases. For example, unmethylated Cs are converted to Ts while leaving methylated Cs unmodified. These processes are cumbersome, error prone, and not generally adaptable to other epigenetic variations.

Pacific Biosciences is developing an optical technique to observe polymerase-mediated synthesis in real time using arrays of zero-mode waveguides. Reads of 10 k to 100 k bases are typical, and epigenetic information is available to some extent based on the timing and duration of polymerase events.

Nanopore sequencing (like that from Oxford Nanopore Technologies) is based on modifications of the current through trans-membrane pores as ssDNA progresses through a nanopore. One difficulty is that existing biological nanopores contain 4- to 6-bases within the narrow part of the pores, requiring a complex machine learning analysis to untangle the identity of individual nucleotides. Further, any epigenetic variations significantly complicate this analysis.

Thus, there remains a need for an accurate sequencing approach. Potential needs are being suitable for long reads (1 kbase to 100 kbases or more); operating on native DNA without the need for amplification; and identifying epigenetic variations.

The molecular vibrational frequencies and relative intensities (spectral fingerprints) monitored by Raman scattering provide unique identification of DNA nucleotides including epigenetic variations thereof. Unfortunately, traditional Raman scattering, performed on bulk samples, is a weak effect requiring large ensembles for sufficient signal, and the volumes accessible (˜wavelength³˜1 μm³), limited by diffraction, are much larger than individual nucleotides (˜0.03 nm³).

Surface-enhanced Raman scattering (SERS) eases these problems. SERS is a local “antenna” effect that 1) provides large enhancements (10⁹ to 10¹¹); and 2) dramatically improves spatial resolution as a result of near-field effects associated with metallic nanostructures. The local near-fields associated with metal nanostructures are much greater (by 10- to 1000-times) than incident fields and are localized to ≲1 nm³ dimensions, much smaller than the diffraction limit and approaching molecular scales. SERS is well established as a single molecule spectroscopic technique. See J. Langer et al., “Present and Future of Surface-Enhanced Raman Scattering,” ACS Nano 14, 1, 28-117 (2020).

The inventors have found that incorporating an engineered SERS enhancement structure (ES) into a nanochannel provides single nucleotide sensitivity and spatial resolution in a long-read format with epigenetic information. Accordingly, this disclosure provides methods and apparatus for controlling the position and motion (including dwell time and velocity) of a polymer molecule (e.g., a long ssDNA molecule) past the hot spot (a localized region of high field enhancement) of an ES. Fabrication methods for the nanofluidic chip comprising the SERS ES are also disclosed. In some embodiments, many parallel nanochannels are readily fabricated and used simultaneously, allowing high throughput scaling of the disclosed methods.

While this disclosure makes reference to ssDNA, the materials and methods disclosed herein are applicable to many different polymers including DNA, RNA, proteins and others. To avoid repetition, this disclosure refers to ssDNA, but a skilled artisan should understand that the disclosure equally applies to other polymers, not limited to those recited above.

### SUMMARY

This disclosure relates to a sequencing system, based on fluid flow of a solution containing a polymer (e.g., ssDNA) through a nanoscale chip containing an enhancement structure (ES) positioned in the nanochannels to allow surface-enhanced Raman scattering readout of the individual polymer units (e.g., nucleotides of ssDNA, including epigenetic variations). In some embodiments, the nanoscale chip includes nanostructures to assist in unravelling the polymer. In some embodiments, low frequency (dc to ˜GHz) electric fields are applied along the channel structure (i.e., longitudinally) to assist in stretching the polymer in the nanochannels and to control the movement of (e.g., advance or reverse) the polymer past the ES. In some embodiments, transverse electric fields are applied to hold the polymer in position while a longitudinal field is applied to stretch the polymer past the ES. In some embodiments, the solution can include materials that pacify the nanochannel walls to improve the fluid flow.

Filling of the nanochannel can be by capillary action, by positive/negative pressure applied at the entry/exit ports, or by electrokinetic effects of an applied electric field. In some embodiments, the surfaces of the ES are passivated to reduce stiction between the polymer and the ESs. In some embodiments, a porous material (e.g., silica beads or mesoporous silica) is introduced into the nanochannel on one or both sides of the ES to slow the polymer's velocity through the nanochannel and allow greater control of the polymer motion past the ES. In some embodiments, multiple nanochannels are fabricated in parallel and multiple polymer molecules are sequenced in parallel.

The SERS signatures of individual polymer units are read as they pass the hot spot of the ES with an external backward geometry Raman scattering apparatus. The apparatus comprises: a source laser; a dichroic beam splitter for separating the reflected pump laser beam and the Raman signals; a long pass filter for additional suppression of the pump laser intensity in the collection arm; and a spectrometer with a high sensitivity detector/camera interfaced with a computer for signal processing and base recognition.

Some embodiments of this disclosure relate to a nanofluidic chip. The chip comprises a nanochannel comprising a SERS enhancement structure. In some embodiments, the enhancement structure is located in a pocket formed in the nanochannel. In some embodiments, the chip further comprises alignment marks for enabling the positioning of a laser excitation beam onto the enhancement structure. The chip further comprises a controller for positioning a polymer within the chip relative to the enhancement structure. In some embodiments, the enhancement structure may comprise a disc of a metal material on a pedestal of an insulator material within the channel.

Additional embodiments of the disclosure relate to a chip reader for sampling the nanofluidic chip. The chip reader comprises: a precision stage for receiving the chip; a source laser; a dichroic beam splitter; a long pass filter; and a spectrometer. In some embodiments, the chip reader further comprises optics which focus the source laser to a linear focal spot. The linear focal spot may extend along a row of enhancement structures. In some embodiments, the chip reader further comprises a processing computer connected to the chip reader for sequencing a polymer within the nanofluidic chip.

Further embodiments of the disclosure relate to fabrication methods for a nanofluidic chip for sequencing polymers. The methods comprise depositing a dielectric film on a substrate. At least one alignment mark, a channel, and a pocket within the channel are formed within the dielectric film. An enhancement structure is formed within the pocket. The channel (and the pocket including the enhancement structure) are covered with a cover slip. In some embodiments, the enhancement structure is formed by depositing an dielectric pillar in the pocket, and depositing a metal film on the dielectric pillar. In further embodiments, the methods include depositing a second dielectric film on the metal film and a second metal film on the second dielectric film.

Other embodiments of the disclosure relate to methods for sequencing a polymer. The methods comprise positioning a polymer within a nanofluidic chip in proximity to an enhancement structure within the chip. A Raman spectrum of a unit of the polymer is recorded. The polymer is moved to position other units of the polymer in proximity to the enhancement structure. The recording and moving steps are repeated to sequence a portion of the polymer. In some embodiments, the methods further comprise utilizing a processing computer to interpret Raman spectra and store the spectra or the interpretations thereof.

### BRIEF DESCRIPTION OF THE DRAWINGS

FIG. 1A illustrates the arrangement of holes in a cover slip and ports etched into a dielectric-coated silicon substrate according to one or more embodiment of the disclosure.

FIG. 1B is a picture of a chip containing two units of the nanoscale chip according to one or more embodiment of the disclosure.

FIGS. 2A-2D are schematics of an optical lithography pattern used to define the input/output regions of a nanofluidic chip according to one or more embodiment of the disclosure.

FIGS. 3A-3B are schematics of a microchannel portion of the interface between the macrochannels shown in FIGS. 3A-3B and the nanochannels shown in FIGS. 4A-4C, according to one or more embodiment of the disclosure.

FIGS. 4A-4F show details of the interface microchannel regions and the entrance to the nanochannels, according to one or more embodiment of the disclosure.

FIGS. 5A-5C are scanning electron micrographs (SEM) of portions of the etched pattern in the nanochannel region, according to one or more embodiment of the disclosure.

FIG. 6 is a SEM of an enhancement structure within a pocket of a nanochannel with input and output nanochannels, according to one or more embodiment of the disclosure.

FIGS. 7A-7B show views of localization of a ssDNA strand at the enhancement structure hot spot, according to one or more embodiment of the disclosure.

FIG. 7C shows a fabricated nanochannel structure with a deep (80 nm) section one side of the pocket and a less deep (40 nm) section on the other side of the pocket, according to one or more embodiment of the disclosure.

FIG. 8 shows a disc-shaped enhancement structure with a thin overcoat of a dielectric material according to one or more embodiment of the disclosure.

FIG. 9 shows reflectivity and field intensity at the surface of a silicon nitride thin film atop a silicon substrate as a function of the film thickness.

FIGS. 10A-10C show views various electrode configurations for manipulation of the polymer, according to one or more embodiment of the disclosure.

FIG. 11A shows nanochannels filled with silica nanoparticles deposited by spin coating.

FIG. 11B is a schematic of filled portions of microchannels with mesoporous silica by EISA.

FIG. 12 is a flow chart for fabrication of the nanofluidic chip according to one or more embodiment of the disclosure.

FIG. 13 shows the alignment marks deposited to assist in the alignment of the laser focal point during operation, according to one or more embodiment of the disclosure.

FIG. 14 shows process sequences for fabricating enhancement structures in pockets according to one or more embodiment of the disclosure.

FIG. 15 shows acquisition of an array of SERS spectra from a polymer at hot spots of a parallel array of enhancement structures and identification of single units, according to one or more embodiment of the disclosure.

FIGS. 16A-16B show multiplex arrangements where multiple enhancement structures are sampled in a single exposure. The multiplexing can be across multiple parallel nanochannels (FIG. 16A) or across multiple pockets and enhancement structures in a single channel (FIG. 16B).

FIG. 17 is a series of consecutive frames of a movie showing dsDNA [lambda phage (48.5 kbases)] propagating through the obstruction regions and through the nanochannels. The top row (A) evidences a 80 nm deep nanochannels and the bottom row (B) evidences 80/40/80 nm deep nanochannels. The circles highlight a single dsDNA molecule that transits a nanochannel.

FIG. 18 shows microscope images of red fluorescently labeled ssDNA attached to green fluorescent beads that are too large to fit into the nanochannels and are captured at the entrance to the nanochannels, according to one or more embodiment of the disclosure.

### DETAILED DESCRIPTION

### Nanofluidic Chip

Before discussing the details of the nanofluidic chip, it is useful to provide an overview. In some embodiments, dielectric-coated silicon substrates are used for the fabrication. Alternatively, glass substrates can be used.

Electron-beam lithography is used to fabricate the central portion of the system, including microchannels, barrier arrays to straighten the ssDNA, nanochannels, pockets in the nanochannels, enhancement structures, etc. If a glass substrate is used, an appropriate conductive film is deposited before the e-beam lithography steps. Optical lithography is used to fabricate the entry/exit ports and larger microchannels that allow input/output of fluids containing DNA from the system.

Enhancement structures for surface-enhanced Raman scattering (SERS) are positioned in pockets along the nanochannels. Electrodes, placed both longitudinally, at the entry/exit ports, and transversely, across the nanochannels, may be used to control the motion of the ssDNA. In one embodiment, interdigitated electrodes are provided for “clocking” of the ssDNA motion. In some embodiments, nanoscale silica beads or mesoporous silica material are used to fill sections of the micro- or nano-channels to provide velocity control of the DNA.

In some embodiments, a glass cover slip, Corning 7740, Borofloat 33, Schott 8830, or comparable glass composition thermally matched to silicon expansion, is bonded over the lithographically defined structures to seal the channels. If a glass substrate is used, an appropriate thermal expansion matched cover slip is used. Holes are machined into the cover slip to align with the lithographically defined input/output ports.

Bonding can be by anodic bonding, chemical/thermal bonding or other alternate techniques. In some embodiments, the surfaces of the final chip are hydrophilic to allow for capillary filling of the fluid. An oxygen plasma treatment of both the patterned substrate and the cover slip immediately prior to bonding is one exemplary approach for ensuring appropriate surface properties.

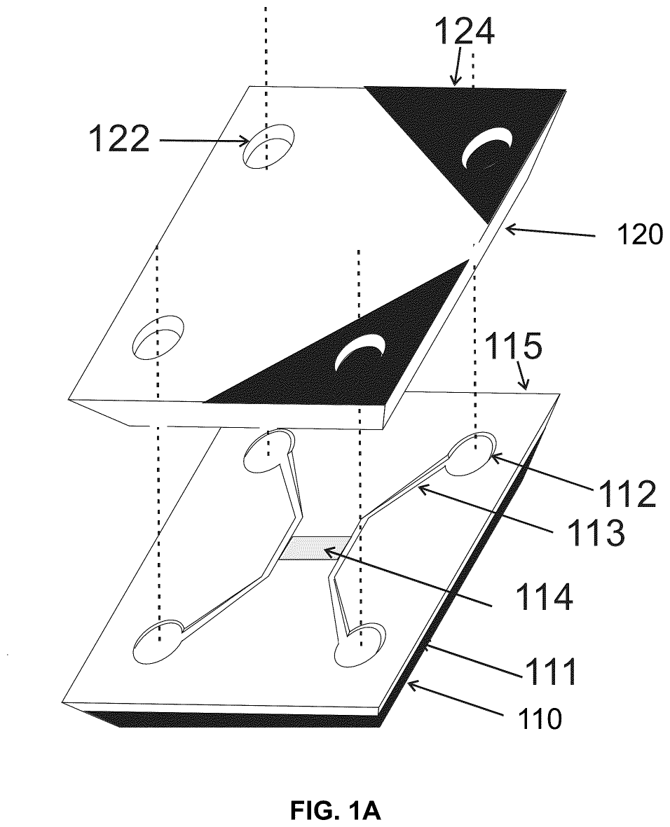

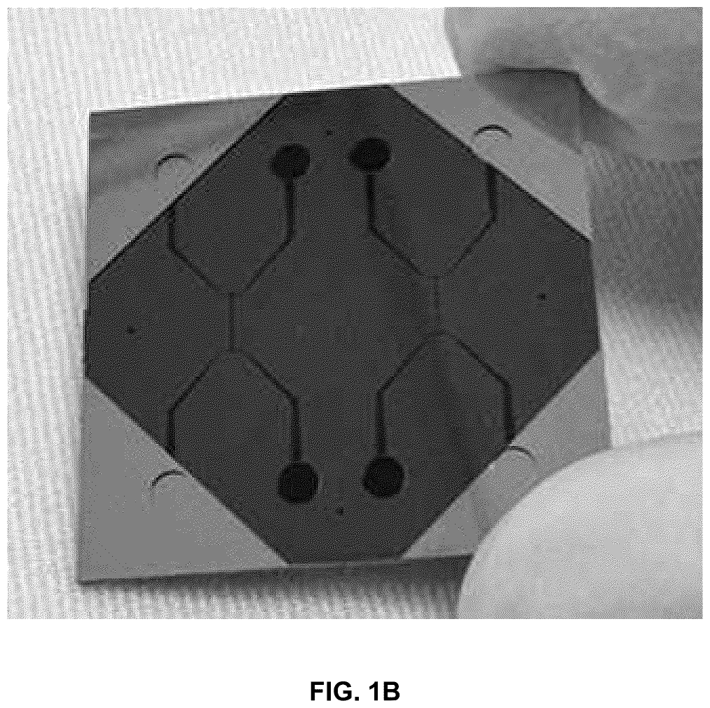

FIG. 1A shows an arrangement of chip features before bonding of the cover slip 120 to the patterned dielectric film. Substrate 110 is coated with a dielectric film 111. In some embodiments, the dielectric film has a thickness ranging from about 100 nm to 1000 nm. Exemplary materials for this film are, but are not limited to, SiO₂; Si₃N₄; TiO₂ and Al₂O₃. As used herein references to SiO₂ and the like are considered to refer to materials containing the referenced elements in their approximate stoichiometry without limit to the absolute ratios disclosed. In one embodiment, a multilayer stack, similar to an interference film stack, is used to enhance the coupling of the incident and scattered electromagnetic fields to the hot spot of the ES.

An electron-beam lithography pattern is produced in area 114 of the dielectric film developed and etched about 50 nm to about 200 nm deep into the dielectric film 111. Details of the pattern in area 114 are presented below.

Following the formation of the etched pattern in area 114, an optical lithography pattern 113 is exposed and developed with larger features, ranging from less than about 100 μm to several mm. This pattern is etched to a depth of about 200 nm to about 700 nm while protecting the electron-beam lithography pattern.

The division of the total pattern between optical and e-beam lithography steps depends on the capabilities of the available lithography tools. Throughput and manufacturing costs favor optical lithography as much as possible. Included in the optical lithography pattern are entry/exit ports 112 which are macroscopic to allow filling by pipette or by tubing connectors. In one design embodiment, these ports are about 1.5 mm in diameter. They may be etched to a depth less than the thickness of the dielectric film to avoid conductivity through the silicon substrate.

The glass cover slip 120 has machined through holes 122, which are larger than the diameter of the entry/exit ports 112 to provide for external addition of fluids. In some embodiments, the dielectric film and the cover slip are may be bonded using anodic bonding (see K. M. Knowles, “Anodic Bonding,” Intl Matl. Rev. 51, 273-311 (2006)) or any other suitable attachment mechanism.

After bonding a platinum evaporation may be utilized to provide an electric contact 124 at the input and output ports. For these embodiments, the circuit is completed when fluids are loaded.

FIG. 1B shows two completed units on a 25×25 mm² silicon-nitride-coated silicon substrate. It is emphasized that alternate manufacturing processes are available including nanoimprint lithography, discussed below.

### Macro- to Micro- to Nano-Fluidic Design

The fluidic transition from macroscale structures (˜1 mm, that allow the introduction of fluid containing the ssDNA to be sequenced) to the nanoscale structures (˜10 nm to 100 nm, that form the nanochannels and the enhancement structures) is a notable element of the system design discussed further below.

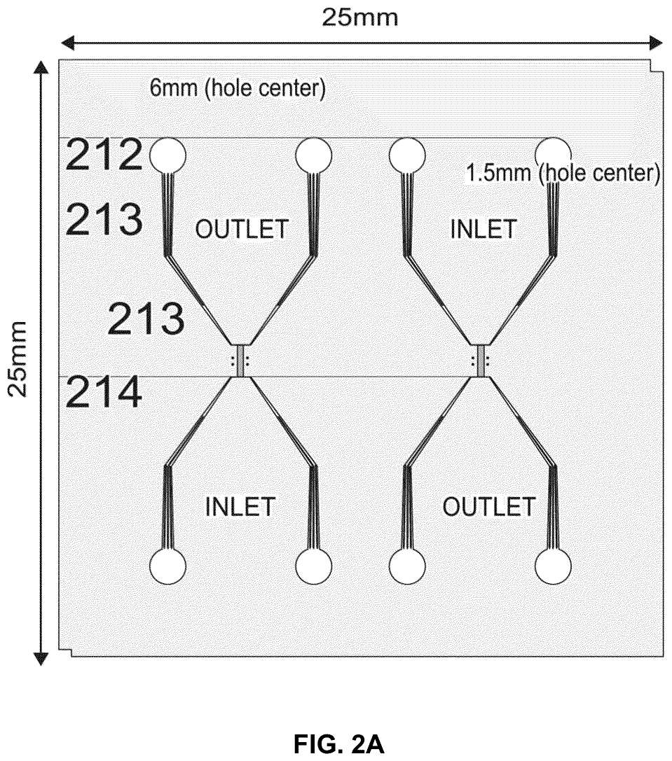

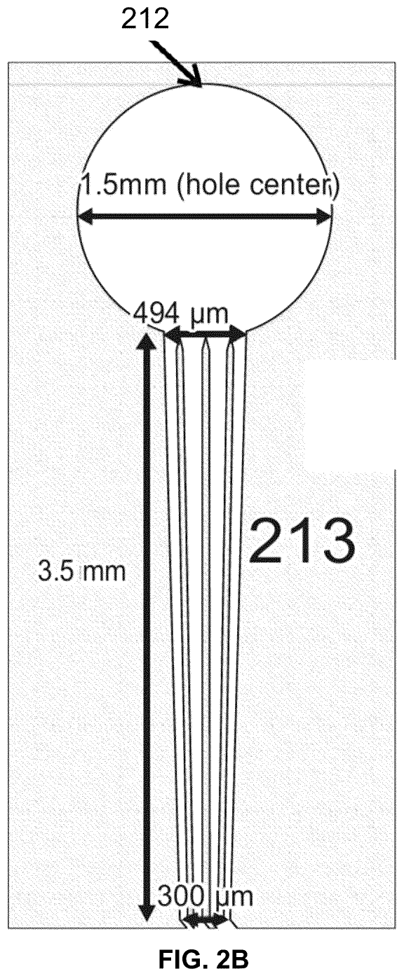

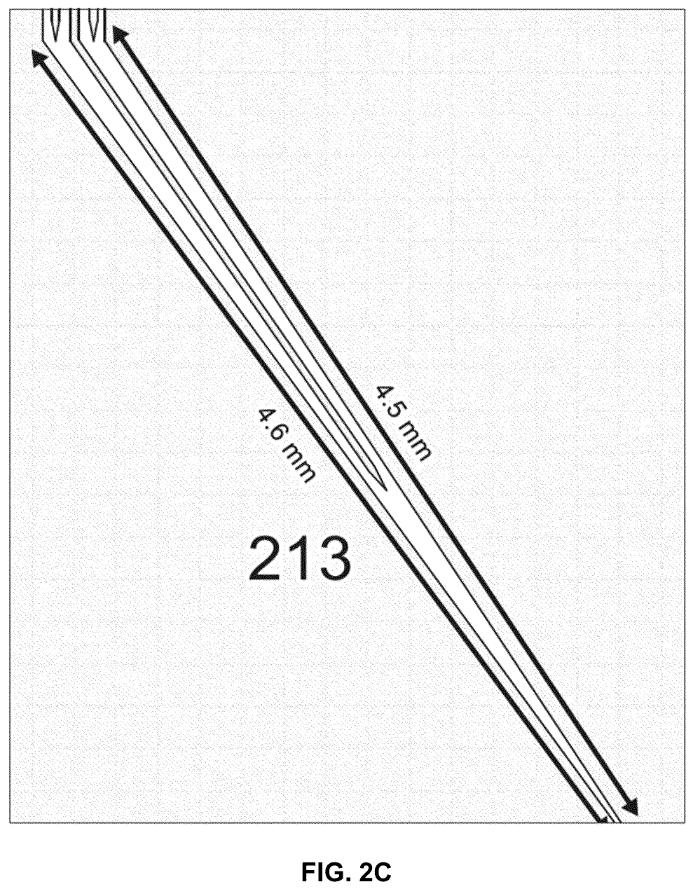

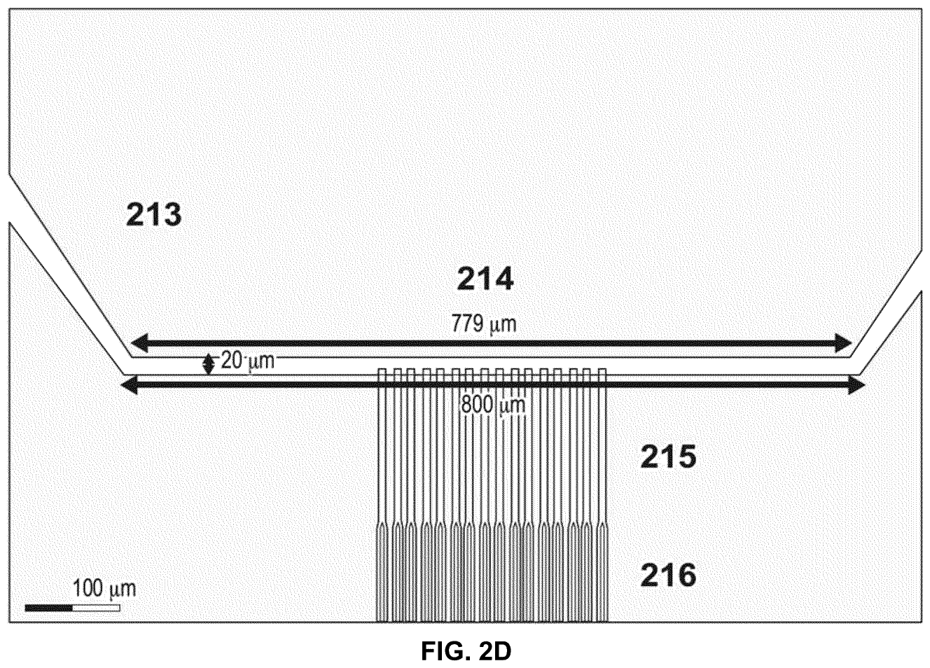

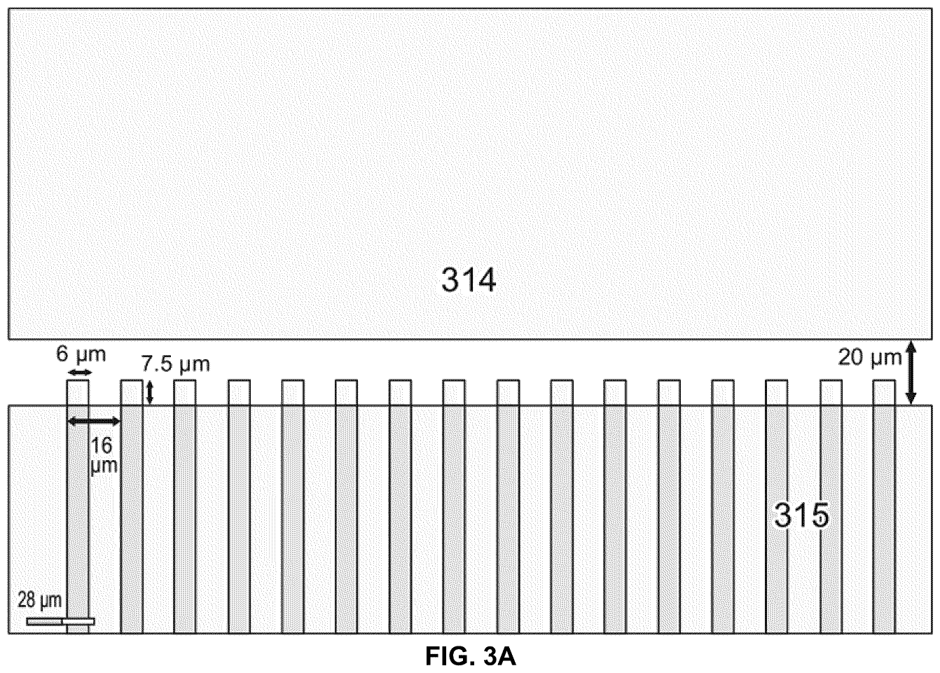

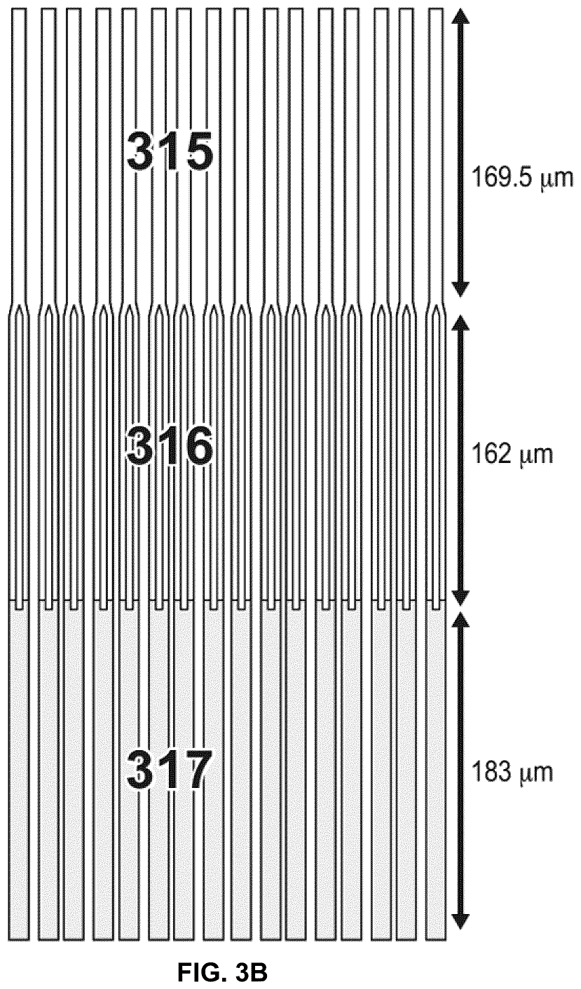

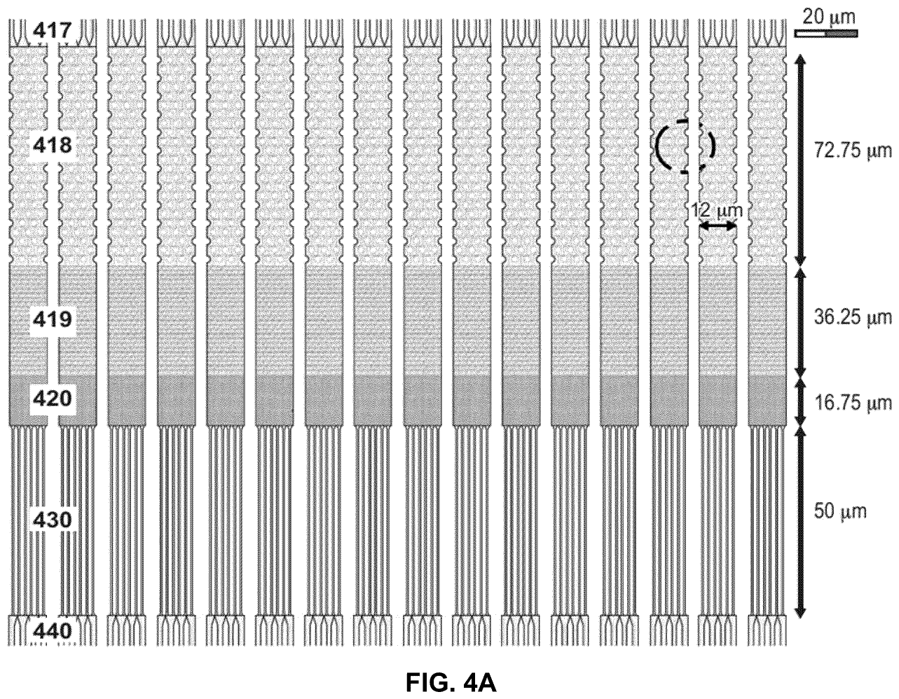

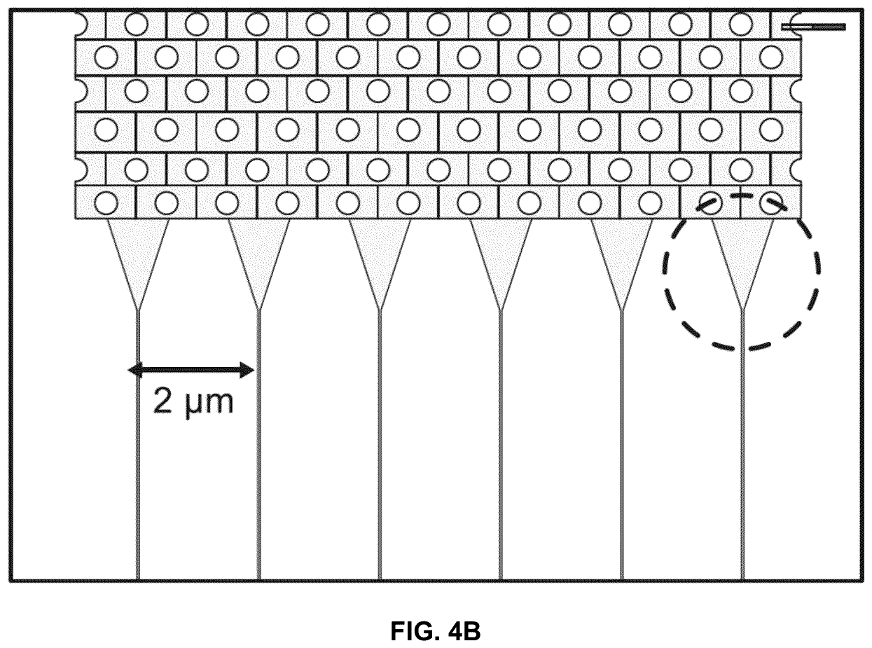

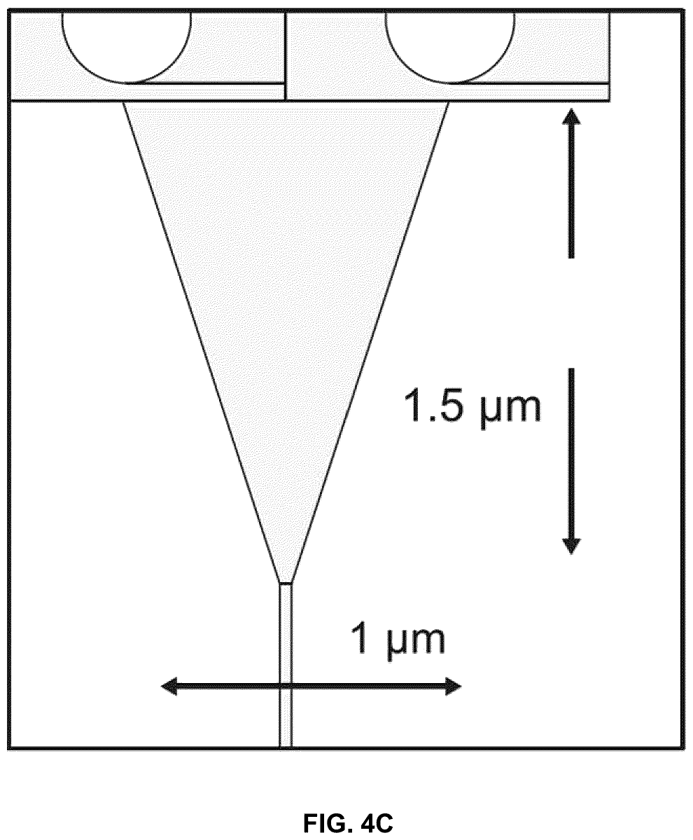

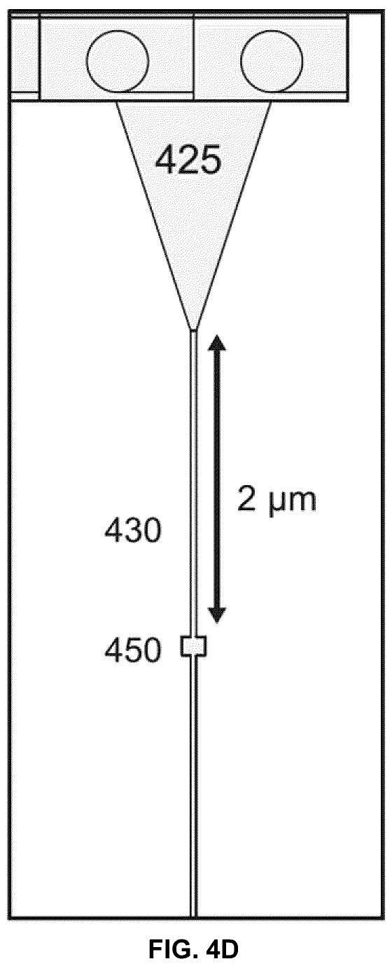

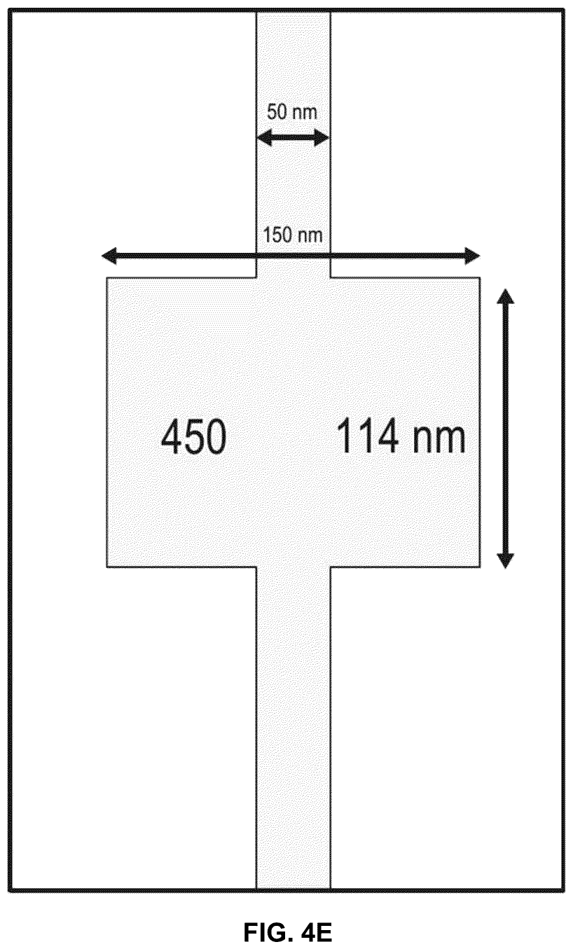

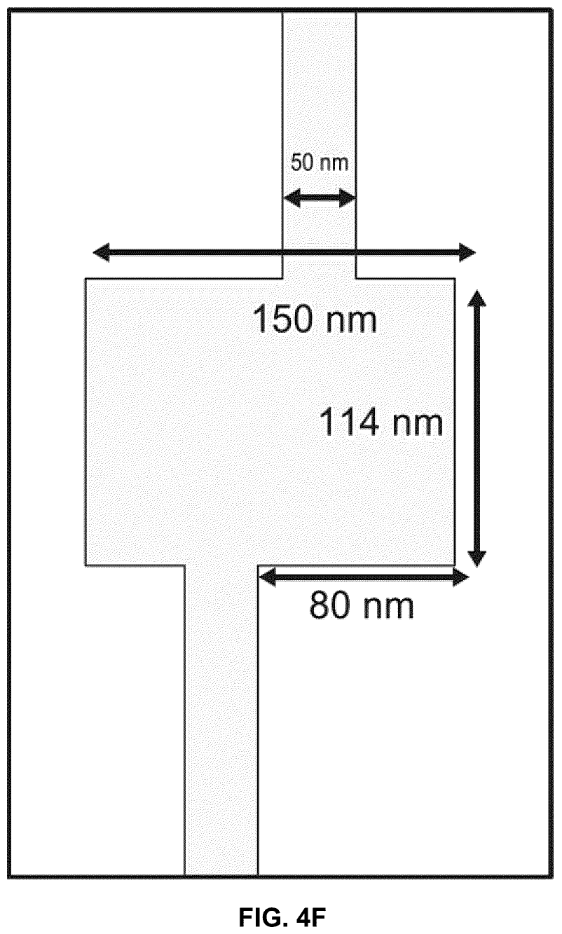

This transition may be accomplished in incremental stages. For clarity, this disclosure relies on separate, but similar reference numerals for identical objects in different drawings. Specifically, a channel labelled as 215 in FIGS. 2A-2D will be labelled as 315 in FIGS. 3A-3B. Similarly, a channel labelled 317 in FIGS. 3A-3B will be labelled as 417 in FIGS. 4A-4F.

FIG. 2A shows two units with the overall pattern of an optical lithography step positioned on a 25×25 mm² wafer. In some embodiments, additional copies may be provided on a larger wafer. It will be understood that the specific dimensions provided in this portion of the description are not critical and changes can be made without deviating from the scope of the disclosure.

In the illustrated example, ports 212 (about 1.5 mm in diameter) are connected by a series of tapered channels (213, see FIG. 2B and FIG. 2C) that feed into a straight channel (214, see FIG. 2D) with a width of 10 μm to 50 μm. FIG. 2A shows two ports 212 for both input and out-put sections. This is useful for introducing buffer/DNA in a T-channel configuration (see Garcia et al., “Electrokinetic molecular separation in nanoscale fluidic channels,” LabChip 5, 1271-1276, (2005).). In other embodiments, only a single port can be used in either the input or output sections. The channels 213, 214 and ports 212 are etched to a depth of about 200 nm to about 500 nm.

Several 2 μm to 10 μm wide microchannels 215 extend from the channel 214 and direct the fluid towards the nanochannel region. In some embodiments, the microchannels 215 are defined in an electron-beam lithography exposure.

In one embodiment, shown in FIG. 2D and FIG. 3A, there are 16 microchannels 215, the width of each of the microchannels is about 6 μm, and the microchannels 215 are etched 80 nm deep. In some embodiments, there is an overlap between the straight channel 214/314 and the microchannels 215/315 to allow for misalignment between the optical and electron-beam lithography patterns. As shown in FIG. 3B, the microchannels 315 are divided in a tree-like configuration as they progress towards the nanochannels, reducing the cross section of each microchannel. In some embodiments, the reduction is by a factor of two at each node, from 6 μm to 3 μm (shown as 316) and then to 1.5 μm (shown as 317).

As shown in FIG. 4A, at the terminus of the last set of microchannels 417, there are wide microchannels containing a post arrays 418, 419, 420 of obstacles. In the illustrated embodiments, the post arrays contain hexagonal posts with diameters of 2.1 μm at 418, 1.1 mm at 419 and 0.4 mm at 420 that assist in straightening out the DNA chains as they flow past this region. While the illustrated embodiments, the post array shows three regions with increasingly smaller obstacles, any arrangement and size of obstacles is within the scope of this disclosure.

Following this obstacle course, as shown in FIG. 4B (an expanded view of the circled region in FIG. 4A) and FIG. 4C (expanded view of the circled region in FIG. 4B), there are 16 groups of 50 μm long and 40 nm to 50 nm wide nanochannels 430, etched about 80 nm deep into the dielectric film. Each group contains 6 nanochannels, for a total of 96 nanochannels per device. In some embodiments, longer nanochannels up to mm scale are used.

At the entry to each nanochannel 430 there is a small funnel 425 to ease the entry of the leading edge of the DNA. The funnel 425 may also serve as a constriction to trap a bead for experiments where the DNA is attached to a bead to anchor the DNA at the start of the nanochannel 430. In some embodiments, the bead is greater than or equal to about 50 nm in diameter.

Pockets 450 for enhancement structures are placed along the nanochannels. In the example shown in FIG. 4D, the pocket is placed about 2 μm from the start of the nanochannel 430 and is 150×114 nm². It will be understood that there is considerable flexibility in the length of the nanochannels 430, the funnel 425 dimensions, the placement and size of these pockets 450, and whether the nanochannels extend straight through the pocket (as in FIG. 4E) or are offset (as in FIG. 4F). In some embodiments, at the exit of the nanochannels 430, there are a set of funnels 425 and nanochannels similar to those on the input side of the nanochannels 430. These exit structures are also flexible and can be modified for different applications.

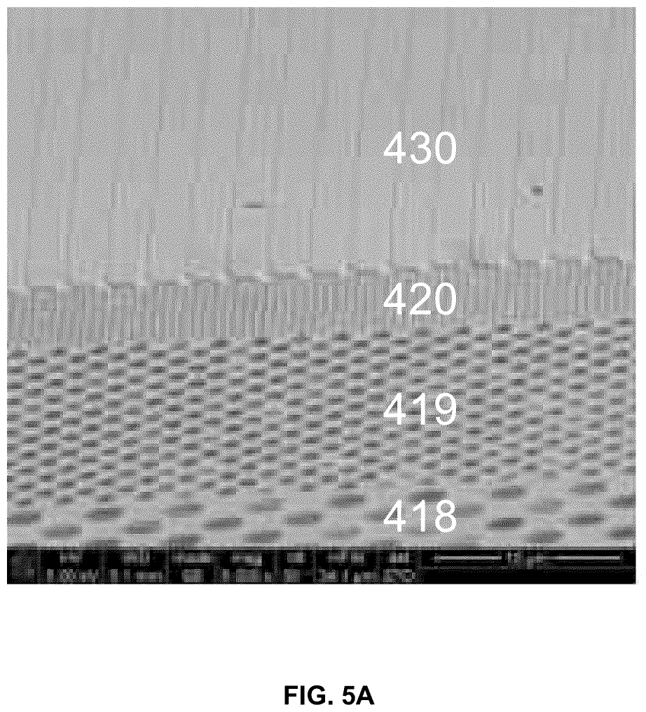

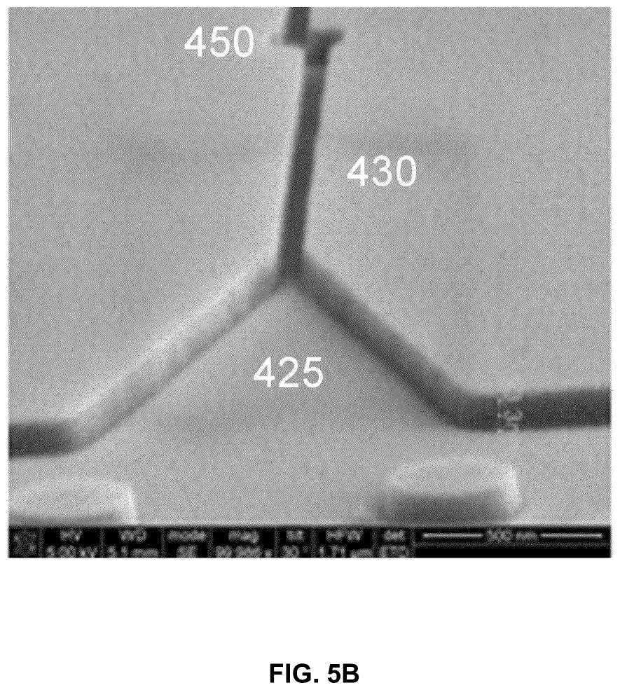

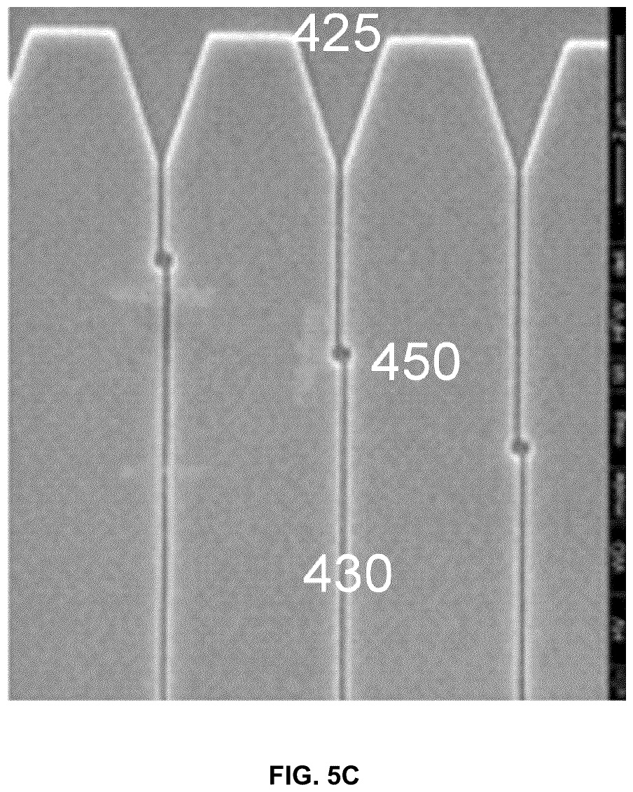

FIG. 5A shows a scanning electron micrograph (SEM) of a portion of an etched post array as well as nanochannels. Visible at the bottom of the SEM in FIG. 5A are three sections of hexagonally placed obstructions with decreasing dimensions 418, 419, 420, the entry funnels 425 and portions of the nanochannels 430. FIG. 5B is a magnified view of an entry funnel 425, a portion of a nanochannel 430, and a pocket 450. As shown in FIGS. 5B-5C, the nanochannel 430 from the entry funnel 425 to the pocket 450 and the nanochannel after the pocket are offset from each other, according to one or more embodiment of this invention. FIG. 5C is a top-down view (SEM) of several entry funnels 425, nanochannels 430 and pockets 450.

### Enhancement Structures

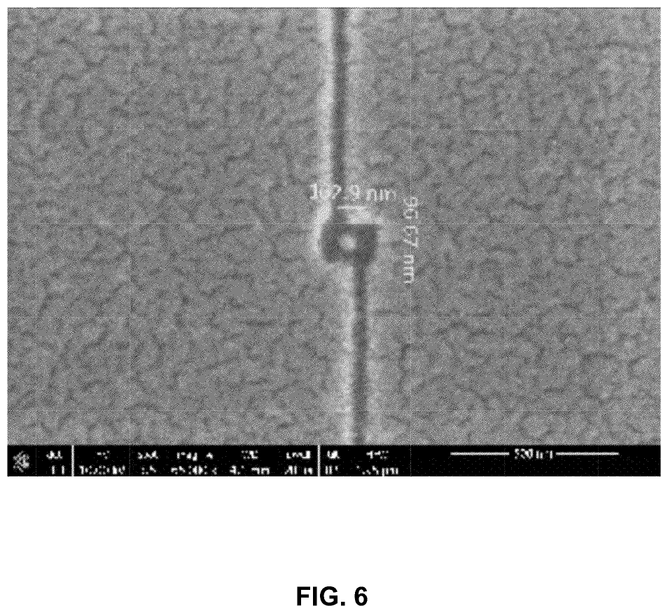

Metal-dielectric enhancement structures (ES) are positioned in the pockets using electron beam lithography and deposition followed by a lift-off step to remove the remaining e-beam resist. The ES can be a metal disc with circular or elliptical cross sections, or any other shape. The metal is selected from silver, gold, or aluminum, although others may also be used. Metal-insulator-metal (MIM) and metal-insulator-metal-insulator (MIM⁺) ES are also envisioned and provides greater enhancements. FIG. 6 shows an ES 660 placed inside a pocket 650 with offset nanochannels 630.

As detailed in a previous patent application (WO 2024/026441), a hot spot with maximum enhancement for a metal-insulator disc-type ES is localized: to less than or equal to about 1 nm radially away from the disc; to less than or equal to about 1 nm vertically from the interface between the metal and the insulator; and is spread circumferentially across a significant spread that extends much further than the approximately 0.3 nm linear dimension of a single DNA nucleotide.

For a disc ES, the total enhancement scales roughly as cos(4Θ)⁴ where Θ=0 is the direction of the electric field of the incident pump optical beam used for the SERS measurement. Thus, the regions of the ES with enhancements within 50% of the peak are a pair of narrow slits just at the interface with the insulator extended circumferentially about ±33° [cos 4(33°)⁴=0.49] each with a length of =dΘ_{radians} about 30 nm or about 100 nucleotides for a nominal d=100 nm diameter disc. This dimension is decreased somewhat with an elliptical structure when the incident beam is polarized (E-field) along the long axis of the ellipse but remains much larger than ssDNA nucleotide dimensions.

Thus, it is necessary to provide guiding elements to the nanochannel pattern to 1) force a ssDNA strand to pass through this hot spot and; and 2) to control the motion of the ssDNA past the hot spot commensurate with the signal acquisition demands of single nucleotide SERS, which depend on the optical system, the Raman cross sections of the individual nucleotides and the strength of the hot spot. According to a simple model, this strength scales as β²(ωₚ)β²(ωₛ) where β(ωₚ) is the electric field enhancement at the pump wavelength and β(ωₛ) is the electric field enhancement at the Stokes (Raman-shifted) wavelength.

### Polymer Position

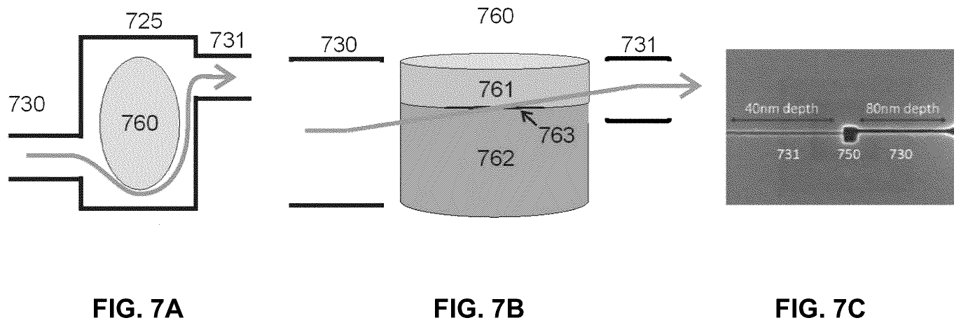

Placing the ES in the pocket and offsetting the input and output nanochannels in the x-y plane (parallel to the substrate) is used in some embodiments to force the ssDNA to pass the ES as shown in FIG. 7A. Additionally, the input and output channels can be fabricated in two lithography steps with different depths as show in the side view (x-z plane) of FIG. 7B. For example, the depth of the input nanochannel 730 could be 80 nm and the depth of the output channel 731 could be 40 nm, resulting in the ssDNA strand to pass the hot spot with a vertical tilt.

This approach has two known advantages: 1) since the hot spot is highly localized in the vertical direction, the tilt of the ssDNA passing the hot spot provides the necessary spatial resolution for monitoring a small number of (preferably one) nucleotides; and 2) since the hot spot is actually an extended “hot line” circumferentially, this geometry assures that the DNA will pass somewhere within the hot line. In this example, the ES structure 760 is composed of a simple gold elliptical disc 761 atop an insulating pedestal 762. The hot spot is denoted as 763. As discussed above the hot-lines are a pair of circumferential arcs at the metal-insulator interface centered along the polarization direction of the incident pump laser beam. In fabrication, the insulating pedestal and the gold disc are self-aligned and are deposited sequentially in a single lithography/develop/deposition/liftoff step.

FIG. 7C shows a SEM of a fabricated nanochannel structure with two different nanochannel depths on the two sides of a pocket structure. Alternative enhancement structures such as metal-insulator-metal elliptical designs are available (and have been disclosed in application PCT/US2023/071183, published as WO 2024/026441). As disclosed in this prior application, the strongest hot spot is along the interface between the metal and the higher index dielectric. Alternate process sequences are disclosed below in the fabrication section for fabrication an “inverted” ES with the strongest hot spot at the top, rather than the bottom, of the metallic portion of the ES. This can allow taller ES such as metal-insulator-metal (MIM) structures within the confines of the nanochannel.

DNA nucleotides exhibit different affinities for adsorption to the metals in the enhancement structures. See Koo et al., “DNA-bare gold affinity interactions: mechanism and applications in biosensing,” Anal. Methods 7, 7042-7054 (2015). This can lead to a sequence-dependent stiction that impacts the motion of the ssDNA past the hot spot and could lead to a sequence-dependent stick-slip motion that interferes with reading of the full DNA sequence.

The addition of a thin dielectric layer over the enhancement structure with a conformal atomic layer deposition (ALD) process provides a method to overcome this effect since the adsorption requires charge transfer effects that operate only with close contact (intermolecular distances) between the metal and the adsorbate (e.g., nucleotide). In some embodiments, this layer comprises silicon oxide, silicon nitride, titanium oxide, aluminum oxide, or any suitable insulating material. In the case of aluminum as the metal, a self-limiting oxide can be formed by controlled exposure to an oxygen ambient. Electromagnetic simulations show that the enhancement is only weakly affected by this extra layer.

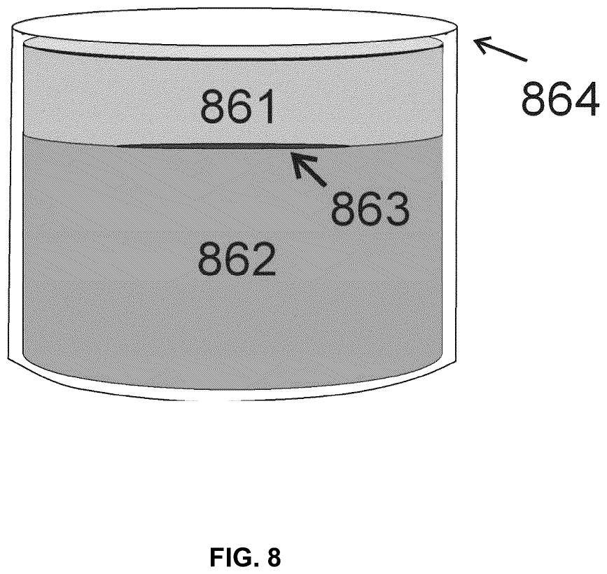

FIG. 8 shows this layer 864 applied to a simple disk on a dielectric pedestal. The layer can be applied in the same process step as the deposition of the ES, e.g., with resist protecting most of the chip except for the ES, or it can be applied at later stages in the fabrication when additional surfaces are exposed to the ALD process.

In addition to the position in the x-y plane discussed above, it is important to place the enhancement structure vertically in an optimal position for SERS. Since the dominant substrate for e-beam lithography is silicon, and since a dielectric film is necessary to provide electrical isolation from the substrate, there are thin film reflectivity effects that have a significant impact on the observed SERS signal levels.

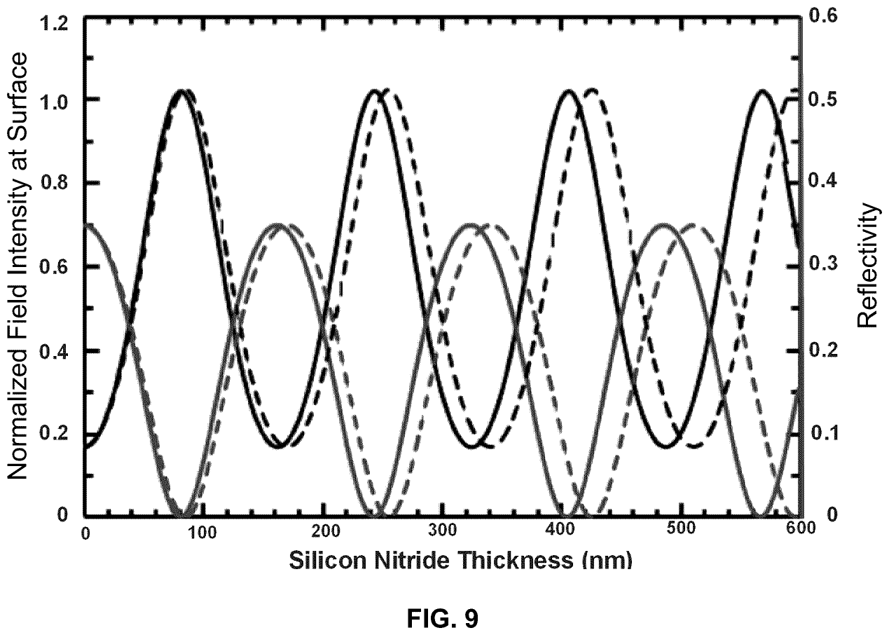

This is most easily understood by investigating the field intensity at the top interface of a thin silicon nitride film atop a silicon substrate as a function of the thickness of the film as shown in FIG. 9, along with the associated far-field reflectivity from the nitride-silicon structure. The two black curves show the field intensity at the metal-insulator interface, e. g. at the hot spot of the ES: the solid curve at a pump wavelength of 633 nm; the dashed curve at a typical scattered (Stokes) wavelength of 666 nm (Raman shift of ˜770 cm⁻¹). The associated reflectivity curves are shown in red.

Silicon nitride is a good anti-reflection coating for silicon (refractive index of ˜2˜\sqrt{3.6}=1.9, where 3.6 is the refractive index of Si), at a thickness of one quarter wavelength in the silicon nitride, the reflectivity is close to zero. The optimum thicknesses for the highest intensity at the surface, and hence the highest SERS signal, coincide with the minima in the reflectivity, since at these thicknesses there is no reflected field—the reflection from the air-nitride interface is cancelled by the out-of-phase reflection from the nitride-silicon interface. The curves for the pump and Stokes wavelength are shifted, but there is a good overlap region where both are large.

This provides the possibility of further enhancements using additional thin films to further increase the field at the surface, similar to the well-known use of film stacks to realize high-reflectivity (HR) and low reflectivity (AR) coatings in optics. In particular, for a film stack of Si₃N₄/SiO₂/Si, there is constructive interference between the various reflected fields at the top surface of the film and the maximum field at the surface is increased to about 1.7 relative to the incident field. Further increases, up to a theoretical maximum of 2 (and therefore about 16× in the SERS signal) are available with further tuning of the film stack.

The addition of nanoscale features, such as the enhancement structures in the pocket and the nanofluidic features will change the details, but not the overall concept. The design of the overall structure is to maximize the electromagnetic intensities at the position of the hot spot at both the pump and Stokes wavelengths. Finite-difference-time-domain (FDTD) or related electromagnetic modeling approaches are well developed and suitable for this modeling.

### Polymer Motion

In addition to controlling the position of the ssDNA as it passes the hot spot, the motion of the ssDNA past the hot spot must be controlled to allow sequencing. SERS at the single-molecule level requires a significant integration time for signal acquisition with adequate signal-to-noise. At present enhancement levels, this time is between 0.1 s and 10 s to allow sufficient signal for high readout accuracy. Improvements in both the optical configuration and the ES will lead to reductions in this acquisition time. Nonetheless, SERS readout will require careful control of the ssDNA motion past the ES hot spot (typical transit times for ssDNA through an artificial nanopore are about 1 μs/base, much faster than Raman readout times).

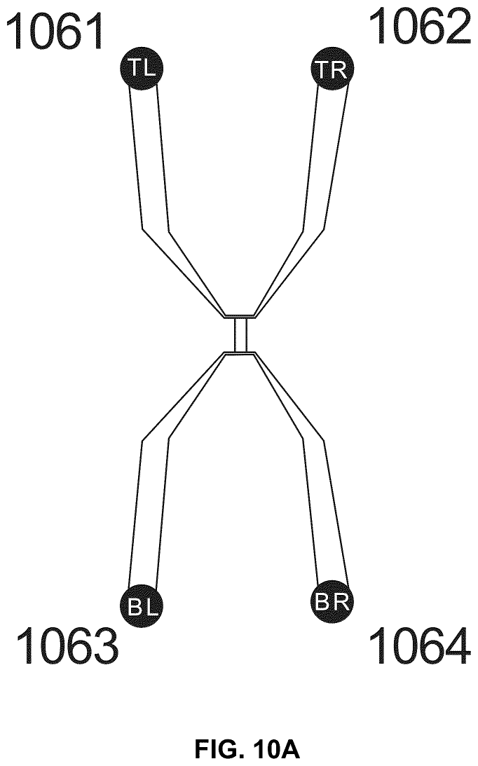

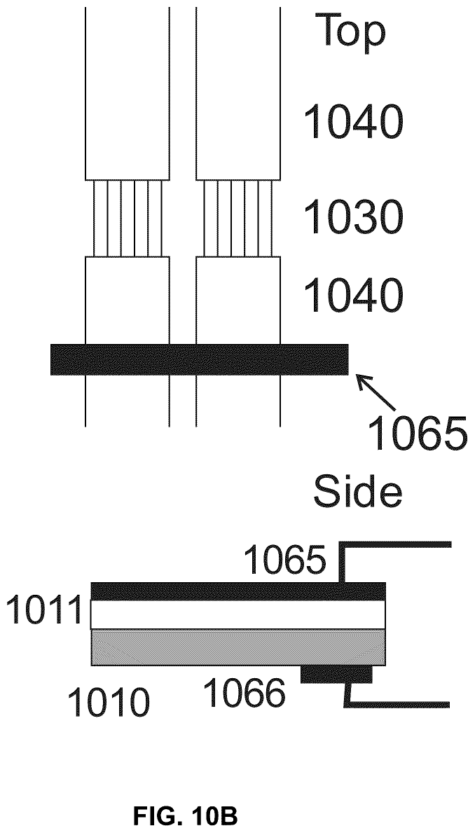

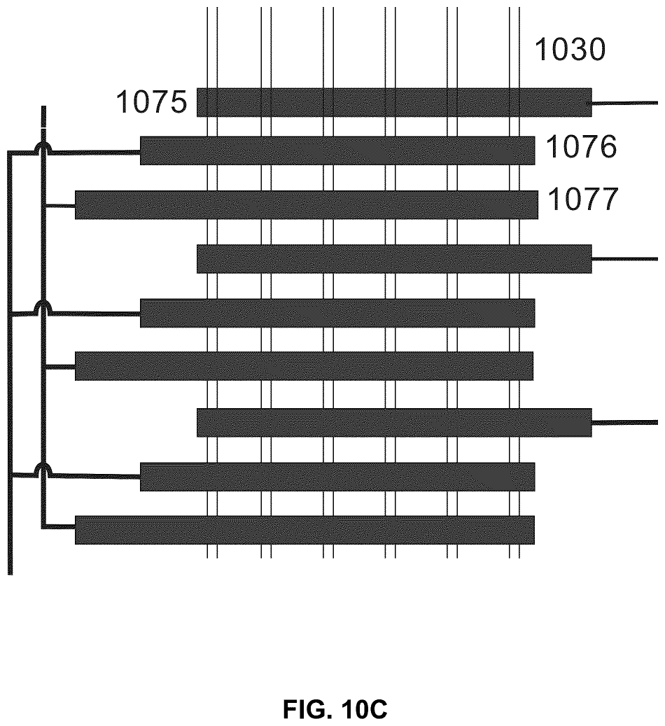

There are several mechanisms available. First is the application of a low frequency electric field. As shown in FIG. 10A, electrodes 1061-1064 are available to apply potentials across the entire structure. These can be used in a conventional T-cell microfluidic approach to move a portion of buffer containing the ssDNA to the entrance to the micro- or nano-channels by applying a voltage between 1061 and 1062 while blocking any flow towards the nanochannels with a bias applied to 1063 and 1064. Then the flow can be switched by switching the voltages to put 1061 and 1062 at the same potential and adjusting the potential of 1063 and 1064 to draw the ssDNA across the nanochannels containing the ES structures in pockets.

This can be a constant (dc) field combined with a pulsed field to assist in clocking the DNA or reversing the motion to “floss” or backup and reread a section as necessary. Other field profiles, such as a sawtooth or a sinusoidal profile are possible and will find use in various applications. The flow will be a combination of electrophoretic (response of the charges attached to the ssDNA) and electroosmosis (flow of the fluid induced as a result of electric field forces acting on the double layer at the channel walls).

An additional electrode can be added above the roof (see FIG. 10B element 1065) and a potential applied between this electrode and the silicon substrate to provide a transverse field to “clamp” the ssDNA. After clamping, the longitudinal field can be varied to stretch the ssDNA for sequencing of a small region.

In one embodiment, this electrode is placed in the nanochannel region, in either the input or output nanochannels surrounding the ES and the pocket. In another embodiment, this electrode is in the microchannel regions feeding into or out of the nanochannels. In yet another embodiment, electrode 1065 is a transparent electrode (e.g., indium-tin-oxide) and is directly over the ES. The silicon substrate must have sufficient conductivity to allow the operation of these field effects. Alternatively, a recess can be etched into the back side of the substrate to bring a counter electrode 1066 close to the nanochannel structure.

As seen in FIG. 10C, Additional functionality can be realized in another embodiment by adding a triplet of interdigitated electrodes 1075-1077. These may be used, in a manner analogous to the clocking of charge in a CCD camera, to advance a section of ssDNA, where the section is defined by the center-to-center distance between adjacent electrodes.

For this to be effective, the interdigitated electrodes have to be integrated onto the bottom side of the cover slip, since the resolution afforded by the interdigitation is lost if the electrodes are on the opposite side of the cover slip (about 170 μm thick away from the nanochannels).

This requires coating the contacts with a dielectric layer and planarizing before any anodic bonding to insulate the electrodes from the fluidics. In some embodiments, the function of electrode 1065, discussed above, can be combined with these electrodes with the counter electrode on the back side of the silicon substrate.

Additional control over the ssDNA motion can be provided by adding porous media to either the nanochannel or the microchannels. This is analogous to the use of a porous gel to separate ssDNA and dsDNA by length, e.g. to slow the velocity of long-chain DNA under electrophoresis by forcing it to snake through a geometrically complex porous medium.

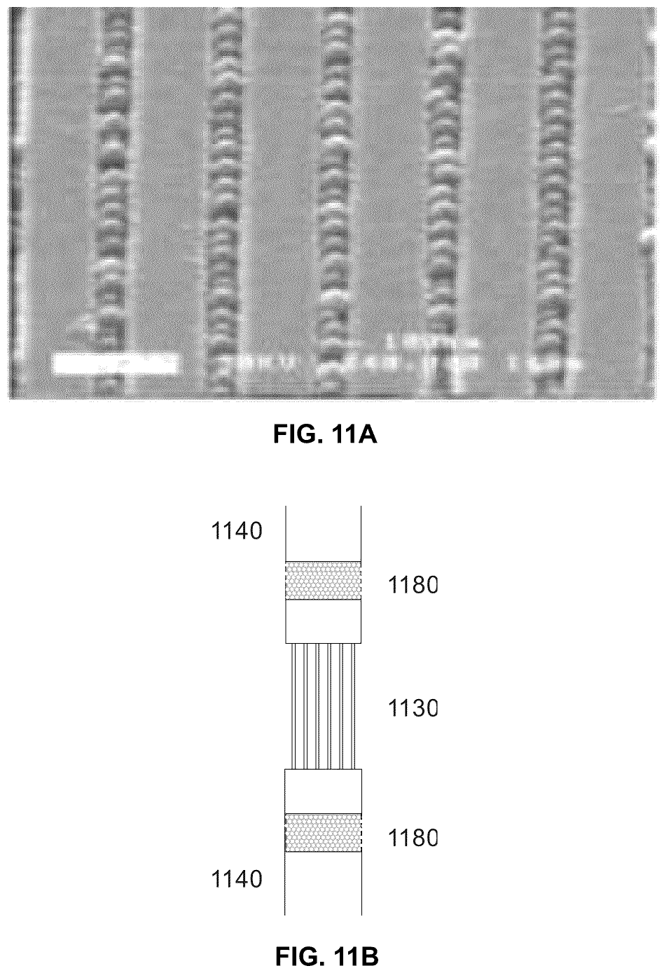

This can be accomplished by spinning-on particles to fill the nanochannels (as shown in FIG. 11A) or the use of a mesoporous silica 1180 formed by evaporation-induced self-assembly (EISA, see M. Ogawa, “Mesoporous Silica Layer: Preparation and Opportunity,” Chem. Rec. 17, 217-232 (2017).) as shown in FIG. 11B.

One advantage of the EISA approach is the uniformity of the pores that is determined chemically rather than by the random and uncontrolled variations in particle size that dominate in the nanoparticle approach. Another advantage of EISA is that the mesoporous silica offers a more uniform top surface that can be fabricated without material protruding above the nanochannel that might interfere with anodic bonding.

In different embodiments, either one section of porous material is inserted in front of the ES and pocket, or two sections are inserted, one before and one after the ES and pocket (as shown in FIG. 11B). Mesoporous silica has been demonstrated to slow the translocation of dsDNA through relatively thin films. In the present case, the length of the propagation through the mesoporous silica will be set lithographically and will be much longer in the mesoporous silica than in previous studies, resulting in substantially increased velocity reduction. See Z. Chen et. al., “DNA translocation through an array of kinked nanopores,” Nature Matls. 9, 667-675 (2010).

### Fabrication of the Nanofluidic Chip

The fabrication sequence presented herein is based on access to a university-maintained user facility which necessarily has a limited set of fabrication tools that are optimized for flexibility and exploratory research rather than for volume manufacturing. In particular, the facilities we access have electron-beam lithography for nanoscale features and an optical lithography system with resolution to only dimensions down to less than or equal to about 1 μm.

Commercial semiconductor manufacturing foundries have access to significantly more capable optical lithography, often to about 20 nm with 193-nm based immersion steppers. EUV lithography and nano-imprint lithography (NIL) provide additional, higher resolution capabilities. Use of these more advanced tools most likely would result in a change in the fabrication sequence to take advantage of the improved lithographic capabilities. Similar comments pertain to other fabrication processes such as deposition, etch and chemical-mechanical polishing. For the sake of definiteness, this application follows the existing fabrication sequence; hereby, modifications based on the availability of well-known semiconductor manufacturing tools are incorporated.

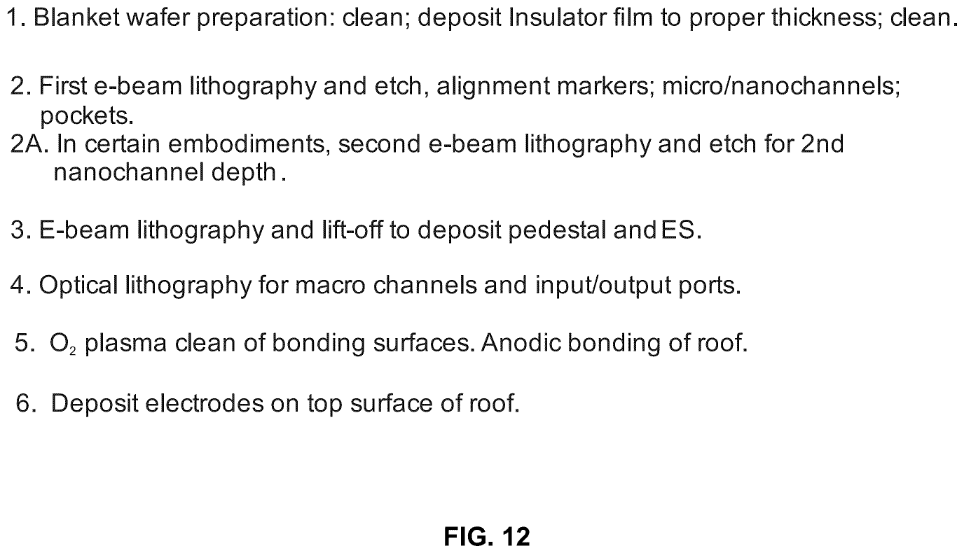

A simplified series of steps for fabrication of the integrated nanofluidic chip is given in FIG. 12. The first steps 12-1 are carried out before any patterning. These consist of cleaning the wafer and depositing blanket insulator film(s) to the proper thickness(es).

Step 12-2 is the first patterning step. In some embodiments, this step includes electron-beam lithography and inductively coupled plasma etching to define the alignment marks that will be needed for subsequent patterning steps, and the micro- and nano-channel regions, including the pockets along the nanochannels. In embodiments with two nanochannel depths (see FIG. 7C), this step is followed by a second e-beam lithography and etch step 12-2A to form additional nanochannels at a second depth.

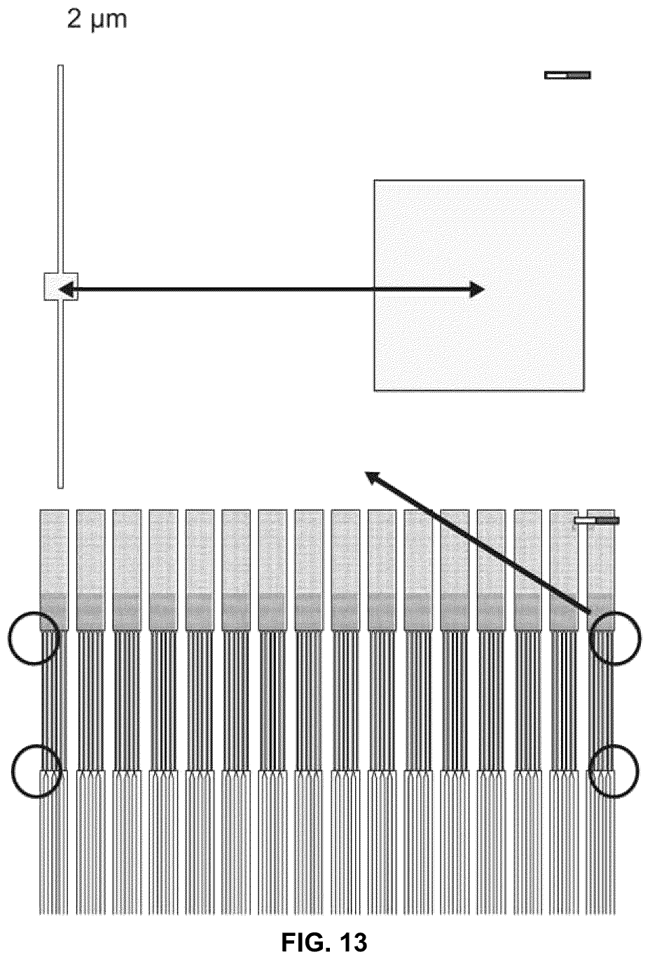

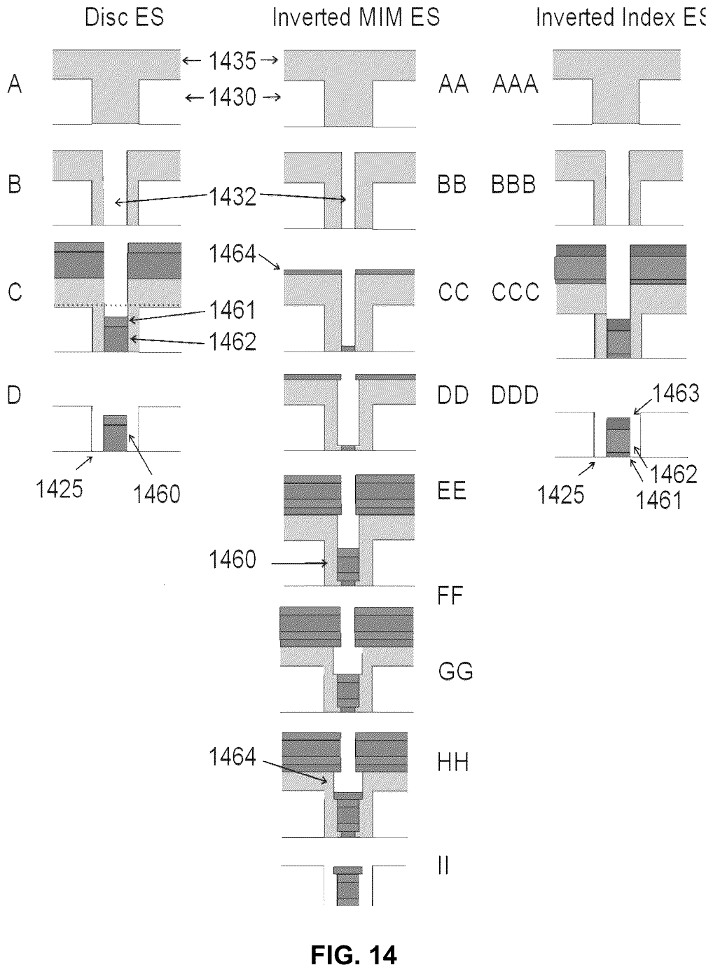

After the processing to form the micro- and nano-channels, a second (or third) electron beam lithography step 12-3 followed by deposition and lift-off is required to deposit the enhancement structures, including an elliptical dielectric pedestal 762 followed by a metal (usually gold) film 761. Alternatives to this enhancement structure are discussed below in conjunction with FIG. 14. In one embodiment, the enhancement structure is coated with a thin dielectric film to using atomic layer deposition to reduce stiction between the metal of the enhancement structure and the long-chain polymer.

In addition to the alignment marks for fabrication, a set of alignment marks are necessary to allow placement of the pump laser onto the pocket areas during measurements. Filling these alignment marks, which are designed to be large enough to accommodate the laser spot and are in line with the pockets of offset, with gold allows monitoring of the laser focal position by the enhanced reflection, and the use of precision stages to move from the alignment spot to the pocket locations. In the current design, there are two alignment marks to each side of the fluidic device (see circles of FIG. 13).

Each mark is 1×1 μm² and is 2 mm away (center to center) from the nearest pocket. These four alignment marks are etched to the same depth as the nanochannel region (e.g., 80 nm). The enhanced reflection from the Au as compared with the surrounding dielectric-film/silicon substrate provides feedback for precise alignment of the laser focal spot.

A precision stage is used to move from the alignment mark to individual ESs. Moving the integrated sequencing chip to all four alignment marks allows orienting the chip to ensure that the stage motion is accurate along and perpendicular to the nanochannels, as required for stepping between ESs. Additional alignment marks can be added as necessary.

Following the e-beam pattern steps, optical lithography is used at step 12-4 to define the macro-channel structure shown in FIGS. 2A-2D. These features are etched deeper into the dielectric film to allow easier loading of the ssDNA. In some embodiments, the etch depth is about 200 nm, in others it can be up to 600 nm. The dielectric film is sufficiently thick so that there is material left after the etching to isolate the electrodes from the silicon substrate.

In a separate processing sequence the glass roof is prepared. This glass can be Borofloat 33 or equivalent that is thermal expansion matched to silicon from room temperature to the bonding temperature of 300° C. to 500° C. This preparation includes drilling of input/output through holes to match the lithographically defined input/output ports. The holes can be 2 mm in diameter on the patterned silicon substrate. If electrodes are incorporated on the bonding side of the glass, they are prepared before the bonding step and through-hole contact vias can be formed in locations that do not interfere with the fluidic arrangements.

In some cases, it is desirable to coat the contact side of the glass cover slip, which forms the nanochannel roof, with a material that can influence the electrokinetics in the fluidic manifold. For example, silicon nitride has a different charge state in contact with the buffer than silicon oxide that influences electroosmosis.

Step 12-5 is the anodic bonding. Immediately before bonding, both the glass roof and the patterned silicon substrate are cleaned by soaking them in piranha solution (3:1 sulfuric acid: hydrogen peroxide) for 15 mins, followed by soaking in DI water for 5 mins, and then rinsing by DI water to remove all the residual acid solution. If gold ES are included in the chip, this cleaning step is omitted since it can affect the Au structures.

The patterned silicon substrate is dehydration-baked on a hotplate at 90° C. for 10 mins. Both the glass roof and the patterned silicon substrate are then oxygen-plasma cleaned in an oxygen asher (Power: 50 W, O₂ flow: 50 mTorr) for 5 mins to create hydrophilic surfaces so that the sealed channels can be filled by capillary forces. The glass and the patterned silicon substrate are brought into contact, and a bond spontaneously forms by gently pressing the glass onto the patterned silicon substrate using a foam swab. The temporarily bonded substrate is then transferred onto a bonding jig with a metal pad over the glass. The temporarily bonded substrate is heated on a hot plate at about 420° C. for 8 mins before applying a high voltage (−800 V to −1000 V) with the cathode contracted to the metal pad and the Si substrate at ground. The voltage is then switched off and the sandwich is allowed to air cool by convection for about 1 to 3 hours. See Knowles et al., “Anodic Bonding,” Intl Matl. Rev. 51, 273-311 (2006) and Dunn et al., “Initiation toughness of silicon/glass anodic bonds,” Acta materialia 48 735-744, (2000).

Finally, in step 12-6 electrodes are deposited on the top surface of the glass to allow electrical contact to the fluid. The order of steps 12-5 and 12-6 can be interchanged without departing from the invention.

### Fabrication of an Enhancement Structure (ES)

The fabrication of a simple disc enhancement structure (ES) is shown on the left side of FIG. 14. The pocket 1425 to contain the ES was formed in the initial electron-beam lithography, develop and etch processes. In FIG. 14-A, a layer of positive electron-beam resist is spun onto the wafer to protect all of the structures. In step FIG. 14-B, electron beam lithography and develop is used to open a hole in the resist at the position and final size of the ES. A light plasma or RIE etch may be necessary to ensure that the resist is cleared at the bottom of the pocket.

In FIG. 14-C, a dielectric spacer is deposited followed by a metal deposition. In one embodiment, the spacer is silicon oxide and the metal is gold, but other materials are possible and advantageous in some applications. The hot spot is at the interface between the silicon oxide and the bottom of the gold disc. Finally, in FIG. 14-D, liftoff is performed to remove any remaining resist and the deposited layers that are not part of the ES. The thickness of the spacer, the thickness of the gold disc, and the shape and placement of the ES are chosen as part of the electromagnetic design optimization to provide as large a SERS enhancement as possible, consistent with fluidic requirements.

The fabrication sequence for an inverted metal-insulator-metal (MIM) ES involves additional steps as shown in the center of FIG. 14. In FIG. 14-AA, the resist is applied to the wafer to fill the pocket as in FIG. 14-A. In FIG. 14-BB, a hole is cleared in the resist for placement of the ES. This hole is the same shape as desired for the ES, but the transverse dimensions are reduced by 10 nm to 30 nm from the final ES dimensions.

A thin, about 5 nm to 10 nm silicon oxide layer is deposited in FIG. 14-CC. Other dielectric materials such as silicon nitride and aluminum oxide may be substituted. This layer will serve as an adhesion layer for subsequent metal deposition. In FIG. 14-DD, an isotropic etch is applied to increase the dimensions of the hole in the resist to the final ES size. This can be accomplished with an isotropic O₂ plasma etch process with reduced oxygen concentration to allow a precision removal of material on the resist sidewalls.

In FIG. 14-EE, the MIM layers are deposited. The next step is a further increase in the dimensions of the resist hole with another precision O₂ plasma etch, as shown in FIG. 14-GG; this increase in dimensions is optional and is used to carefully tailor the hot-spot strength.

This is followed by another dielectric deposition in FIG. 14-HH and liftoff in FIG. 14-II. The dielectric can be one of silicon oxide, silicon nitride, aluminum oxide, or another suitable dielectric.

The total thickness of all the depositions is chosen to match the etch depth of the pocket to ensure that the fluid flow is to the sides of the ES and not over the top. Slightly larger total thicknesses can be accommodated by the anodic bonding step.

The figures are not to scale, in particular, the resist thickness is reduced for clarity, as long as the divergence of the deposition is sufficiently large, the small overhangs shown in FIGS. 14-DD and 14-HH will not affect the sidewall fidelity of the deposition and liftoff. This divergence is a function of the area of the source and the distance from the source to the wafer and can be adjusted as necessary.

Another alternative for an inverted enhancement structure is to deposit a higher index material, such as titanium oxide over the ES as shown in FIGS. 14AAA-DDD. In this embodiment, the hot spot is at the interface between the gold 1462 and the high index material 1463.

These approaches can be combined, adding the material asymmetry of FIG. 14DDD to the geometric asymmetry of FIG. 14-II. One advantage of both of these inverted approaches, individually or combined, is that it provides more vertical space for the gold disc or the MIM compared with the approach in FIG. 14D within the constraints of placing the hot spot at the middle of the nanochannel or at the midpoint between the two nanochannel depths, and of keeping the entire structure extending above the top surface of the nanochannel and interfering with the roof bonding.

### External Apparatus for Raman Measurement

After fabrication, the sequencing chip is mounted onto a carrier which provides for contact to all electrodes and allows handling and mounting into a microscope apparatus for Raman measurements. For a silicon substrate, the Raman scattering apparatus is a standard backward scattering design with a laser source focused onto the wafer. Provision is made for both monitoring reflection of the pump laser for alignment purposes, and fluorescence of labeled DNA for monitoring DNA motion in the fluidic manifold.

Multiple source wavelengths are available. There is a trade off in that shorter wavelengths provide higher Raman cross sections (˜λ⁻⁴), but also give rise to an increased fluorescence background. Resonance enhancement is available for UV wavelengths that vary for different nucleotides, but at the cost of significantly greater fluorescence intensity.

In present experiments the laser wavelength is either 633 nm or 785 nm; but this is not a restriction on the invention. The laser source is a single transverse mode to allow tight focusing to illuminate only a single ES. The laser source can be multi-longitudinal mode as long as the laser line width is narrower than the linewidth of individual Raman peaks from the ssDNA nucleotides.

The alignment marks defined during the electron-beam lithography steps in tandem with the nanochannels and pockets, along with precision stages, allow positioning of the pump laser focal spot over the ES. Polarization control in incorporated to align the polarization of the incident light with the long axis of the elliptical ESs to access the highest Raman signal. High numerical aperture (NA) objectives to collect as much scattered light as possible, either air- or water-immersion are used in conjunction with dichroic beam splitters to separate the incident beam from the Raman scattered light.

A long-pass filter is used to block reflected pump light from propagating in the collection arm. The scattered light is focused onto a spectrometer slit. A cooled low-noise camera is used to detect the scattered light at the output of the spectrometer. This camera is interfaced to a computer for signal processing and nucleotide calling.

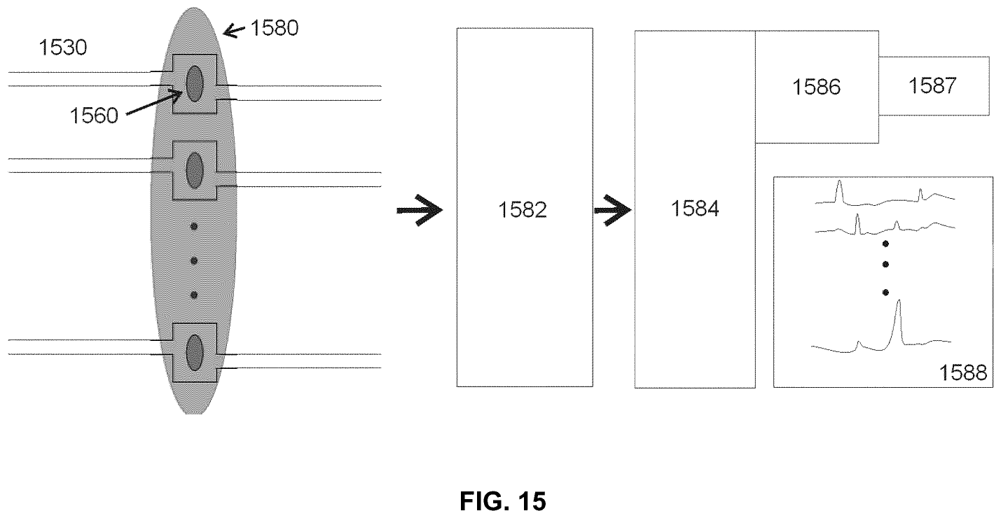

In one embodiment, the laser is focused to a line 1580 at the positions of multiple ESs 1560 in multiple parallel channels as shown in FIG. 15. The scattering line is then focused onto the slit of the spectrometer with an optical system 1582. After passing thru the spectrometer, multiple spectra are available at the focal plane of the camera 1586. These are interfaced with a computer 1587 and spectral analysis software that associates the individual spectra with DNA nucleotides 1588, including epigenetic variations.

This allows monitoring of the SERS signal from multiple enhancement structures, in multiple nanochannels in parallel. The separation between channels has to be sufficient to allow independent spectral analysis of scattering from DNA in each channel. This separation should be larger than 1 μm to 5 μm to allow for adequate separation of the collected radiation from individual channels.

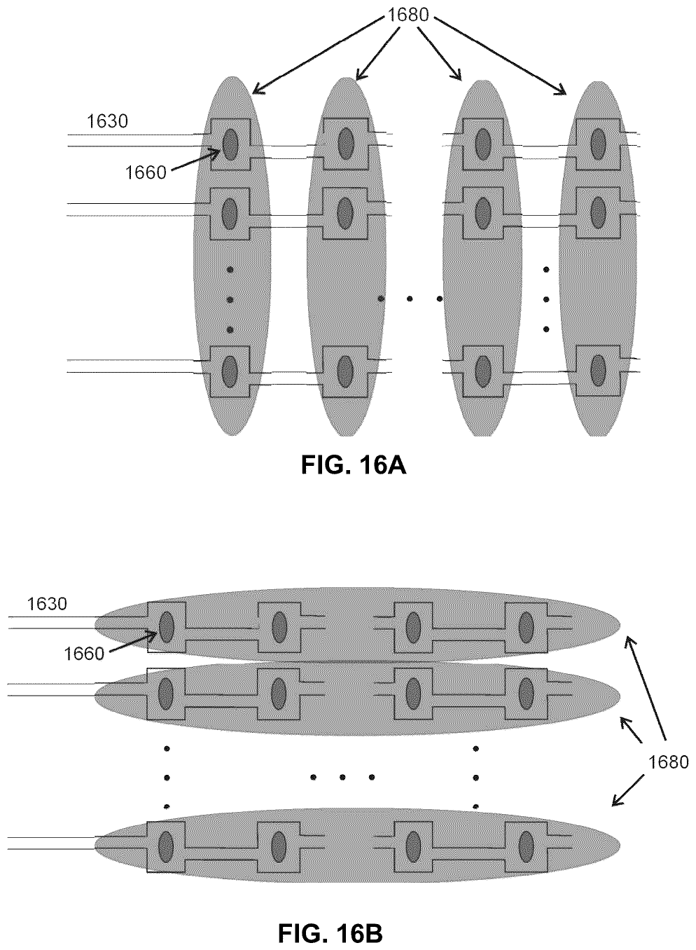

In another embodiment, additional pockets and enhancement structures are placed in a single nanochannel, allowing monitoring of the sequence of the ssDNA at multiple sites along the strand providing redundancy and improved sequencing accuracy. With appropriate optics to avoid spatial overlap of spectra, the multiplexing along a nanochannel and for multiple parallel nanochannels can be combined with excitation slits perpendicular to the nanochannels (FIG. 16A) or along the nanochannels (FIG. 16B).

### Method for Sequencing

In operation, a buffer solution [1× TE (10 nM Tris containing 1 nM EDTA) and 0.1% Tween; pH 8.0] is introduced into the chip by capillary action. There are many variations on this buffer that may be substituted depending on the materials of the nanochannel and the long-chain polymer under investigation. Optionally, the ssDNA is included along with this buffer.

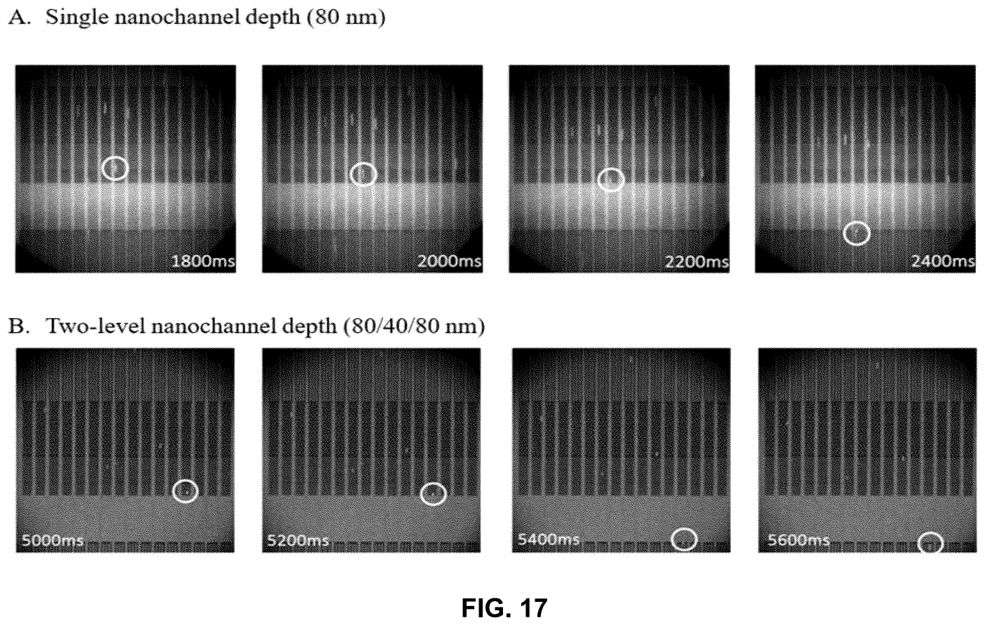

FIG. 17 shows a series of consecutive frames (200 ms integration time, 200 ms intervals between frames) of microscope images of lambda dsDNA dyed with POPO intercalated dye moving through the micro-channel region prior to entering the nanochannels. The motion through the nanochannels is too fast to capture in this experiment. The top row is for single level (80 nm deep) and the bottom row for double level (80/40/80 nm deep) nanochannels. The circles in each row highlight a single DNA molecule as it progresses though the obstacle region and through a nanochannel.

Since the buffer solution is an incompressible fluid, and the cross-sectional area of the microchannels (12,000×80 nm) is much larger than the cross-sectional area of the nanochannels (6×40×80 nm), the velocity through the nanochannels is ˜50× faster than in the obstacle regions, and the transit through the nanochannels is not captured in these images.

Alternatively, the ssDNA, with additional buffer, is added after the chip is filled, and electric fields applied across the input ports are used to move the ssDNA into position above the microchannels. The field is then switched to move the ssDNA through the microchannels and into the nanochannels.

Motion of the ssDNA is by a combination of electrophoresis (the electric field acts on the charges bound to the ssDNA along with solvation charges to move it in the direction of the field) and electroosmosis (electric field acts on the boundary layer between the fluid and the walls of the channels and the ssDNA is dragged along with the fluid in a direction opposite to the field, for negatively charged silicon dioxide walls). The interplay between these two effects is complex and is affected by the ionic concentration of the fluid and by the screening of an applied DC field that result from the many variations in fluidic design (cross section) along the full entry to exit pathway. See Schoch et al., “Transport phenomena in nanofluidics,” Rev. Mod. Phys. 80, 839-883 (2008).

The use of pulsed electric fields, switched on a time scale fast compared to ionic motion across the longitudinal dimensions of the chip structure can offer additional flexibility since the ionic species in the fluid cannot move fast enough to alter the longitudinal screening, ensuring that a field is applied across the length of nanochannels.

Attaching the ssDNA to nanoscale beads, by well-known techniques (see Huang et al., “Binding of biotinylated DNA to streptavidin-coated polystyrene latex,” Anal. Biochem 222, 441-449 (1994)), either silicon or polystyrene, or other materials, that can navigate the microchannels but are two large to fit into the nanochannels provides a mechanism for trapping the DNA and stretching it into the nanochannels and past the ESs.

Then the DNA is fixed at the position of the hot spot, allowing long integration times for SERS data acquisition and, by varying the longitudinal field around the hot spot, the DNA can be stretched to allow sequencing of a local region. See Yeh and Szeto, “Stretching of tethered DNA in nanoslits,” ACS Macro Lett. 5, 1114-1118 (2016).

The trapping of ssDNA attached to beads is illustrated in FIG. 18. FIG. 18A shows red fluorescence from ssDNA bound to polystyrene beads (green fluorescence FIG. 18B) that are trapped at the entry to the nanochannels. FIG. 18C shows the overlap indicating that the about 6 μm contour length ssDNA is stretched into the nanochannels and past the ES in the pockets located 2 μm from the entry funnel.

In addition to the longitudinal voltages applied between the entry/exit ports, transverse voltages applied vertically across the micro/nanochannels can be used to stop the DNA motion, with the major advantage over the bead technique that the ssDNA can be released, advanced, and re-constrained allowing long-read sequencing.

In particular, the ssDNA can be “clamped” in place by the creation of a strong transverse field by applying a voltage between electrodes 1065 and 1066, and released periodically, in tandem with longitudinal electric field pulses, to advance the ssDNA past the hot spot. The position of the electrodes for this transverse field relative to the position of the ES, depends on the length of the ssDNA strand under investigation.

The interdigitated electrodes 1075-1077, along with a counter electrode in the silicon substrate (not shown), provide additional control of the ssDNA motion. If all of the electrodes are biased either positively or negatively, these electrodes are equivalent to the clamping electrode 1065 and the DNA is forced either to the top or to the bottom of the nanochannel. By adjusting the voltages differentially on the three interdigitated arrays, it is possible to “clock” the ssDNA by one electrode spacing, in a manner similar to the readout of a CCD array. Since these electrodes can be positioned with about 50 nm to about 100 nm spacing (i.e., about 150 to 300 nucleotides), this allows very fine clocking of ssDNA in many parallel channels. With the addition of the longitudinal field to stretch the ssDNA past the ES hot spot, it is possible to sequence sections and to reverse the DNA motion and re-sequence for improved accuracy.

1-52. (canceled)

53. A method for sequencing of a long-chain polymer comprising:
positioning a polymer within a nanofluidic chip in proximity to a hot spot of an enhancement structure induced by optical excitation of the enhancement structure in a nanochannel;
recording a Raman spectrum of a moiety of the polymer;
moving the polymer to position sequential moieties of the polymer in proximity to the hot spot of the enhancement structure; and
repeating the recording and moving steps to sequence a portion of the polymer.

54. The method of claim 53, wherein positioning the polymer comprises filling a volume of the chip with a fluid comprising a buffer solution containing specimens of the long-chain polymer between two electrical contacts such that an electrical current is passed along the fluid in the nanochannel between the two electrical contacts.

55. The method of claim 54, wherein the electrical contacts are positioned near entry and exit ports of the chip, creating a longitudinal electric field along the nanochannel.

56. The method of claim 53, wherein moving the polymer comprises using a time varying electric field to move the polymer.

57. The method of claim 53, wherein the electrical contacts are positioned on opposite lateral sides of the enhancement structure, creating a transverse electric field across the nanochannel.

58. The method of claim 53, wherein moving the polymer comprises: anchoring the polymer upstream of the enhancement structure; and stretching the polymer past the enhancement structure.

59. The method of claim 58, wherein anchoring the polymer comprises attaching the polymer to a bead with a diameter larger than the transverse dimensions of the nanochannel.

60. The method of claim 58, wherein anchoring the polymer comprises applying a transverse electrical field.

61. The method of claim 58, wherein stretching the polymer past the enhancement structure comprises applying a longitudinal electrical field.

62. The method of claim 53, wherein moving the polymer comprises: applying a longitudinal electric field to stretch the polymer; and modifying the applied longitudinal field to move the polymer upstream and downstream within the chip.

63. The method of claim 53, wherein moving the polymer comprises powering interdigitated electrodes to advance the polymer in sections.

64. The method of claim 53, further comprising applying a transverse electrical field at the enhancement structure to clamp the polymer for sequencing.

65. The method of claim 64, wherein the transverse electrical field is applied using a transparent electrode.

66. The method of claim 53, wherein moving the polymer comprises modifying the rate of flow through portions of the chip using a porous material to slow the polymer.

67. The method of claim 53, wherein recording the Raman spectrum comprises aligning a focused laser source to one or more enhancement structures with the use of alignment marks on the chip; and imaging the scattered light from the long-chain polymer interacting with the hot spot of the enhancement structure onto the entrance slit of a spectrometer.

68. The method of claim 53, wherein recording the Raman spectrum comprises aligning a line focus of a laser source to an array of enhancement structures; and imaging the scattered light from the long-chain polymers interacting with the hot spots of the illuminated enhancement structures onto the entrance slit of a spectrometer.

69. The method of claim 68, wherein the array of enhancement structures is positioned in parallel channels of the chip.

70. The method of claim 68, wherein the array of enhancement structures is positioned along a single channel of the chip.
